(19) United States
(12) Patent Application Publication
(10) Pub. No.: US 2021/0343020 A1
(43) Pub. Date: Nov. 4, 2021

(54) METHOD FOR SEGMENTING TEETH IN RECONSTRUCTED IMAGES

(71) Applicants: CARESTREAM DENTAL LLC, Atlanta, GA (US); TROPHY SAS, Croissy-Beaubourg (FR)

(72) Inventors: Jay S. SCHILDKRAUT, Rochester, NY (US); Shoupu CHEN, Rochester, NY (US); Jean-Marc INGLESE, Bussy-Saint-Georges (FR); Vincent LOUSTAUNEAU, Fontenay sous Bois (FR)

(21) Appl. No.: 17/284,493

(22) PCT Filed: Oct. 11, 2019
(86) PCT No.: PCT/US19/55760

§ 371 Date: Apr. 12, 2021

Related U.S. Application Data
(60) Provisional application No. 62/744,945, filed on Oct. 12, 2018.

(51) Int. Cl. G06T 7/11

(52) U.S. Cl. CPC G06T 7/11; G06T 2207/30036; G06T 2207/10116

(57) ABSTRACT

The present disclosure describes methods for improving semi-automatic and/or fully automatic tooth segmentation in reconstructed images of X-ray scans using multi-energy X-ray spectra and/or a multi-energy X-ray scanner at more than one energy. Such improved segmentation of teeth in a reconstructed image of an X-ray scan is a critical first step in the utilization of the image for applications in orthodontics, endodontics, and implant planning In accordance with the methods, tooth segmentation may be performed semi-automatically or automatically for images which are reconstructed from a multi-energy X-ray scan. The results of the tooth segmentation may be represented as an image map which identifies voxels which are within a tooth or as a three-dimensional (3D) grid or any other representation of a three-dimensional (3D) spatial region.

### FIELD OF THE INVENTION

The present invention relates to the field of X-ray imaging and, more particularly, to using multi-energy X-ray scans to segment teeth in a reconstructed image.

### BACKGROUND OF THE INVENTION

The segmentation of teeth in a reconstructed image of an X-ray scan is a critical first step in the utilization of the image for applications in orthodontics, endodontics, and implant planning. Teeth segmentation identifies the voxels that belong or correspond to teeth in a three dimensional (3D) reconstructed image of an X-ray scan. More specifically, teeth segmentation may identify a part of the image that comprises teeth, identify individual teeth in the image, and identify parts of a tooth in an image. Different dental applications require different levels of segmentation and it is highly desirable that teeth segmentation be as automatic as possible, requiring little or no human interaction.

Unfortunately, teeth segmentation in reconstructions of X-ray scans is very difficult. Currently, it is not possible to segment teeth fully automatically in all cases. The reason for this is generally two-fold. First, it is difficult to distinguish between tooth roots and surrounding alveolar bone because they have similar material composition. Second, reconstructions have artifacts due to beam hardening, the presence of metal, and scatter which cause material of uniform material composition to appear non-uniform in a reconstructed image.

Therefore, there is a need in the industry for improved semi-automatic and fully automatic methods for segmenting teeth in reconstructed images of X-ray scans that solve the difficulties described herein and other related difficulties.

### SUMMARY OF THE INVENTION

Broadly described according to example embodiments, the present invention comprises methods for producing a three-dimensional (3D), segmented representation of one or more teeth using multi-energy X-ray spectra and/or a multi-energy X-ray scanner. In accordance with the methods, tooth segmentation may be performed automatically or semi-automatically for images which are reconstructed from a multi-energy X-ray scan. The results of the tooth segmentation may be represented in a number of ways. For example, the tooth segmentation results may be represented as an image map which identifies voxels which are within a tooth. Alternatively, the tooth segmentation results may be represented in the form of a three-dimensional (3D) grid or any other representation of a three-dimensional (3D) spatial region.

The example embodiments herein describe the present invention in connection with a dual X-ray spectrum scanner and dual X-ray spectra, but other example embodiments include the use of multiple X-ray spectrum scanners and more than two X-ray spectra. Therefore, the scope of the present invention is not limited to a dual X-ray spectrum scanner or dual X-ray spectra. A dual energy scan can be performed by changing the source voltage and/or filtration of the X-ray source during the scan (fast switching) or performing two separate scans with different source voltage and/or filtration. Alternatively, a dual energy scan may be performed simultaneously with two different sources and detectors. One example embodiment of the present invention includes and uses an energy discriminating photon counting detector with at least two energy bins.

The measured data of an X-ray scan is the exposure value of each pixel of the detector. The exposure value is related to the X-ray attenuation as the photons travel along a line from the source to the detector. The measured data is generally corrected for X-ray source non-uniformity and detector response (flat field correction) and detector defects before it is used for image reconstruction. The measured data at all detector pixels is often referred to as an X-ray projection because it is a radiographic projection of an object onto the detector. A scan consists of a series of projections at different source and detector locations. Often the source and detector move about an axis-of-rotation (AOR). The patient is positioned so that the AOR is located at the center of a region-of-interest (ROI). For dental applications, the ROI is usually within the dental arch. In the dual energy case, two sets of projections are collected. The two projection sets may be for the same or different X-ray paths (source/detector locations).

Advantageously, the methods of the present invention provide the ability to process the scan data before or during the tooth segmentation process so that segmentation can be performed with little or no human intervention. This is accomplished at least in part and alone or in combination by reconstructing the dual energy data in a way that increases the contrast between a tooth and other material in the scanned object such as soft tissue and bone, by reducing artifacts that are caused by the change in X-ray spectrum as it propagates through the scanned material (beam hardening), by reducing artifacts that are due to photon starvation and beam hardening caused by the presence of metal and other dense material, and by reducing artifacts that are caused by X-ray scatter.

Also advantageously, the methods of the present invention include combining the measured scan data at the two X-ray spectra or combining the reconstructions of the scan data at the two X-ray spectra in order to provide an image which is better suited for tooth segmentation than separate reconstructions at each of the two X-ray spectra. Furthermore, the methods provide an ability to incorporate the dual energy scan data into the teeth segmentation process so as to optimize the utility of the dual-spectral data.

Other advantages and benefits of the methods of the present invention will become apparent upon reviewing the detailed description of the example embodiments included below.

### BRIEF DESCRIPTION OF THE DRAWINGS

FIGS. 1A, 1B and 1C display pictorial images of a slice of a reconstruction of a tooth along with a contour that corresponds to the outline of a segmented region.

FIGS. 2A and 2B display pictorial images of a slice of a reconstruction of a tooth having a metal filling.

FIG. 3 displays a schematic diagram of an x-ray scanner positioned relative to a patient.

FIG. 4 displays a flowchart representation of a method for tooth segmentation in accordance with a first example embodiment of the present invention.

FIG. 5 displays a flowchart representation of a method for tooth segmentation in accordance with a second example embodiment of the present invention.

FIG. 6 displays a flowchart representation of a method for tooth segmentation in accordance with a third example embodiment of the present invention.

FIG. 7 displays a flowchart representation of a method for evaluating the quality of tooth segmentation according to an example embodiment of the present invention.

FIG. 8 displays a flowchart representation of a method for determining if under-segmentation is present in the tooth segmentation results.

### DETAILED DESCRIPTION OF EXAMPLE EMBODIMENTS

The methods of the present invention is described herein with respect to a number of example embodiments and with reference to the drawings in which like numerals correspond to like elements or steps throughout the several views. It should be understood and appreciated that while the methods of the present invention is described with respect to various example embodiments, the methods of the present invention may be present and utilized in other example embodiments.

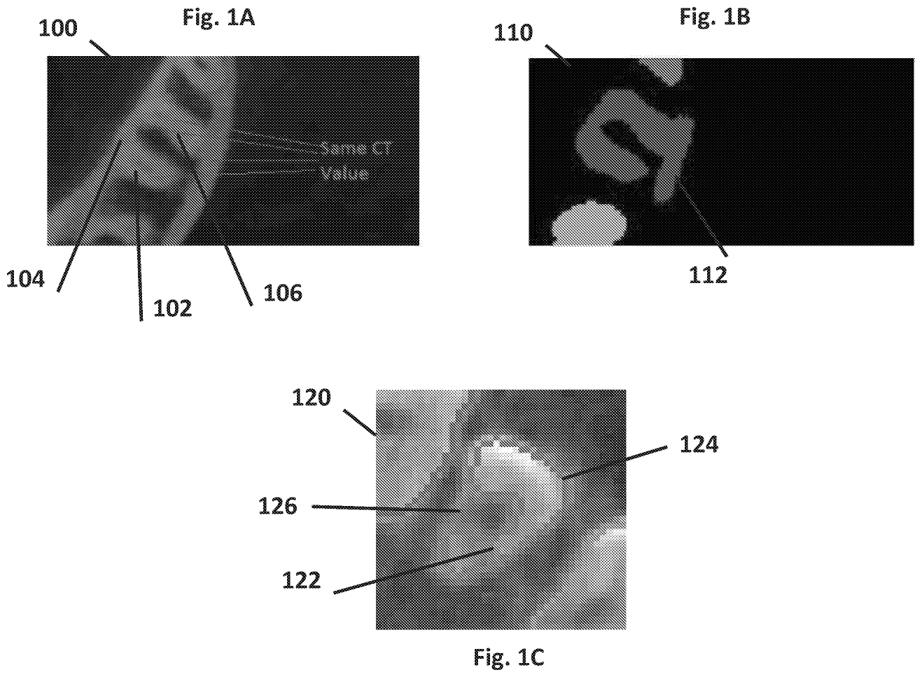

FIGS. 1A, 1B, and 1C display images 100, 110, 120 illustrating two problems that are solved by the methods of the present invention. Image 100 is a slice of a reconstruction of a cone beam scan of a dental arch. Tooth root 102 and surrounding bone 104 appear identical in this image. Image 110 shows the result of segmenting tooth root 102. Because of the inability to distinguish between root 102 and bone 104, the segmentation fails and the segmented region includes not only root 102, but also bone 104 and roots of an adjacent tooth 106.

Image 120 in FIG. 1C shows a slice of a reconstruction with tooth 122 along with contour 124 which is the outline of the segmented region. Region 126 of the tooth is missing from the segmented region because of imaging artifacts which cause tooth 122 to appear non-uniform in the reconstruction. In this case, the image artifact may be caused by beam hardening.

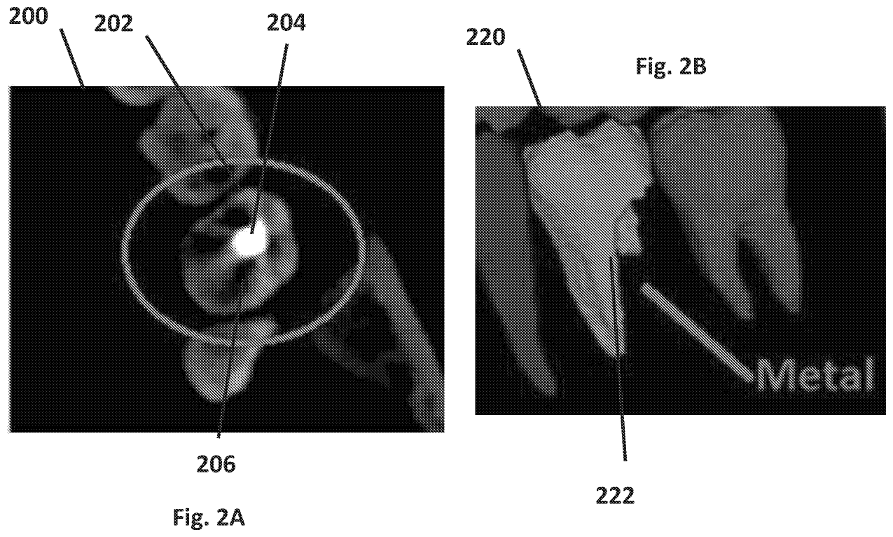

Referring to FIG. 2, image 200 is a slice of a reconstruction having a tooth 202 which contains a metal filling 204. The dark areas in the tooth 206 are an artifact which is caused by the metal filling. Image 220 shows a three-dimensional (3D) representation of the results of segmenting tooth 202 and adjacent teeth. The segmentation of the tooth 222 is missing at least one root because of the metal artifacts present in the reconstruction.

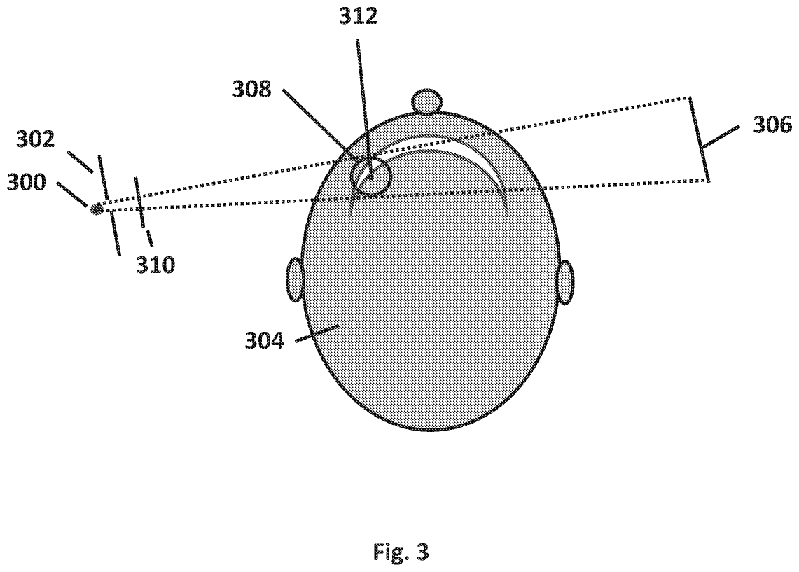

FIG. 3 displays an X-ray scanner. X-rays from source 300 pass through collimator 302 and filter 310. The filter 310 modifies the X-ray energy spectrum and can be used, along with modification of the source's voltage, to choose the X-ray spectrum. The X-rays pass through dental arch region-of-interest (ROI) 308 in the patient's head 304 and are incident on detector 306. To perform a scan, often the source and detector are rotated about AOR 312.

However, other source and detector trajectories are sometimes used. In the case where the detector 306 is an energy discriminating photon counting detector, the dual energy scans are actually a single scan. Otherwise, the voltage of source 300 and filter 310 is changed within a single scan or by performing two scans. The essential outcome of a dual energy scan are two sets of projections for different X-ray spectra which can be used to reconstruct a three dimensional (3D) image of a ROI.

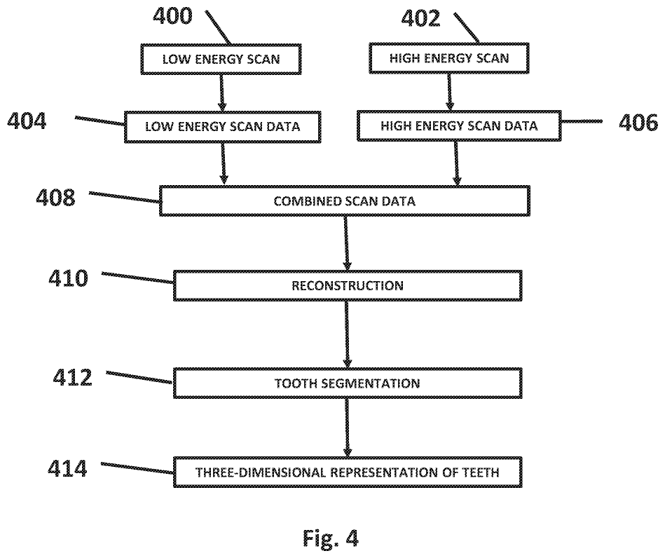

One example embodiment of this invention is shown in FIG. 4. For the purpose of describing this invention, the dual energy scan is described at a low energy scan 400 and a high energy scan 402. This means that the average X-ray photon energy of scan 400 is lower than scan 402. In the case of a scan with an energy discriminating photon counting detector, scan 400 is the photon count in the low energy bin and scan 402 is the photon count in the high energy bin. The low energy scan data 404 and high energy scan data 406 are combined in step 408. The purpose of the step 408 is to combine the low and high energy scan data so that when the data is reconstructed in step 410, the reconstruction has reduced artifacts and increased material contrast. For example, the low energy a_{L} and high energy a_{H} scan data may be combined using a polynomial function,

$${p}_{1} = {\sum }_{i = 0}^{I} {\sum }_{j = 0}^{J} {c}_{ij}^{1} {a}_{L} {a}_{H}$$

$$and$$

$${p}_{2} = {\sum }_{i = 0}^{I} {\sum }_{j = 0}^{J} {c}_{ij}^{2} {a}_{L} {a}_{H}$$

where the coefficients of the polynomial Cᵢⱼ are chosen to enable tooth segmentation step 412.

In step 408, the low and high data may be combined in several different ways. Specifically, the data is combined to enhance the contrast between tooth roots and surrounding alveolar bone. The data may be combined in another way to enhance the contrast between tooth and soft tissue such as the surrounding gum. In one example embodiment, p₁ and p₂ correspond to line integrals of material density for two basis materials. Preferred basis materials for image decomposition are soft tissue and hydroxyapatite, although other materials can be used.

It should be understood that contrast between different materials is not only related to the difference in code values of the materials in the reconstruction, but also to the variation and noise in the code values of each material. One measure of the contrast between two materials is the Mahalanobis distance between the distribution of code values of the materials.

The combined scan data 408 is used in step 410 to create reconstructions that are artifact reduced and preferably artifact free. In one example embodiment of the present invention, this reconstruction is a virtual monochromatic reconstruction meaning that it appears as if it is reconstructed from a scan using a monochromatic X-ray source. Such a reconstruction is free of beam hardening artifacts. Also, the monochromatic energy can be set to maximize the ability to differentiate between materials such as tooth, bone, and soft tissue to enable the subsequent segmentation step 412.

In step 412, one or more teeth in the reconstruction are segmented. This means that each tooth is distinguished from surrounding bone and tissue and from other teeth. This may also include segmenting individual parts of a tooth including crown, enamel, dentin, neck, pulp, and root. This step may use any image segmentation method including neural nets, clustering, active contours, snakes, thresholding, and level sets. The result of this step is a three-dimensional (3D) representation of teeth 414 which may take the form of a three-dimensional (3D) image mask, a surface map, a mesh, or any other means of representing a region in space.

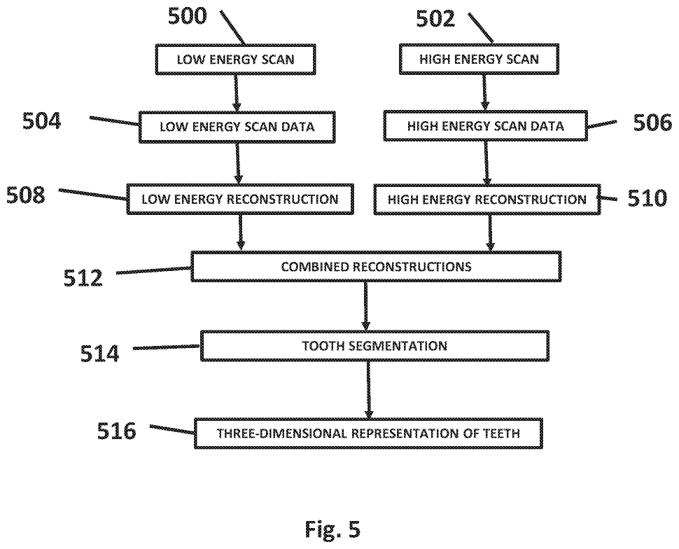

FIG. 5 shows another example embodiment of the present invention. This example embodiment of the invention is most appropriate when the low and high energy scans correspond to different X-ray paths through the object. The low energy scan 500 produces low energy scan data 504 and high energy scan 502 produces high energy scan data 506. The low energy scan data is reconstructed in step 508 and the high energy scan data in step 510. In step 512, the low and high energy reconstructions are combined in order to facilitate the tooth segmentation step 514 which results in a three-dimensional (3D) representation of teeth 516.

In the application of X-ray scans for dentistry, often only a ROI, which is generally located within the dental arch, is scanned. Only this ROI appears in all projections and can be fully reconstructed. Another way of describing this situation is that the X-ray projections are truncated because the projections would need to be larger in order to image all of the scanned object. In this situation, many of the methods of reconstruction artifact reduction including beam hardening correction, scatter removal, and metal artifact reduction are difficult to apply because part, and often most, of the scanned object is unknown although it contributes to artifacts because X-rays pass through for at least some of the projections.

The methods of the present invention use multi-energy scans to improve tooth segmentation, even in the case of truncated projections, by including a way to evaluate the quality of tooth segmentation and to feedback the results into the step in which scan data or reconstructions at two or more energies is combined so that the processing of the scan data and/or reconstruction can be modified in order to facilitate tooth segmentation.

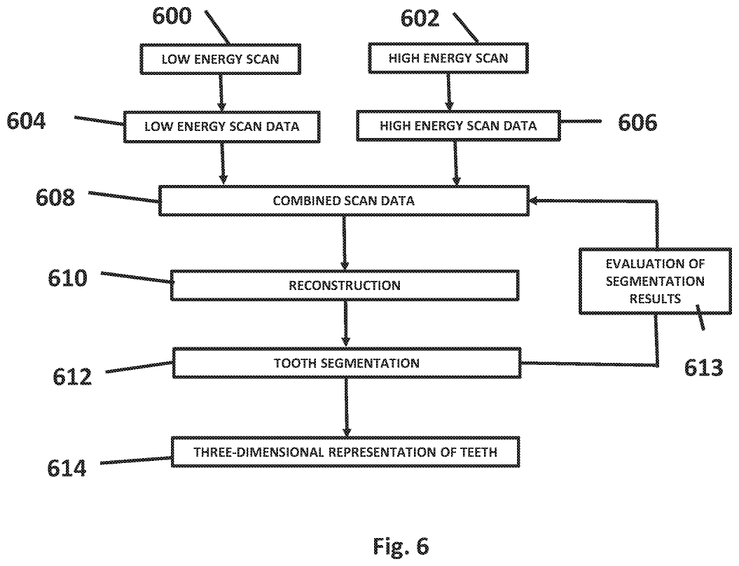

Referring to FIG. 6, a low energy scan 600 and high energy scan 602 are performed to generate low energy 604 and high energy 606 scan data. The combined scan data is processed in step 608 and reconstructed in step 610. The quality of tooth segmentation in step 612 is evaluated in step 613 and the results are input to step 608 in which the scan data is reprocessed in order to improve the segmentation results.

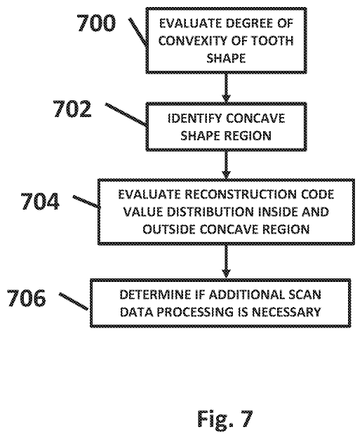

Step 613 can take many different forms. Two example embodiments are described in detail below, but the essence of this step is to provide a measure of teeth segmentation quality. Step 613 may include several quality measures. FIG. 7 illustrates an image uniformity quality evaluation method that determines if the teeth in a reconstruction are being properly segmented. If not, steps 608 and 610 are modified, for example, to improve beam hardening and metal artifact correction.

FIG. 7 displays an example of the steps that occur within step 613. These steps are based on the fact that teeth are usually convex in shape. If the segmentation results are concave this indicates that the dual energy scan data was not sufficiently processed to remove artifacts. In step 700, the degree of convexity of the segmented teeth is calculated. If the convexity is sufficiently low, then the segmentation process is complete and no further processing is necessary. Otherwise, in step 702 concave regions are identified. Contour 124 in FIG. 1B corresponds to an example of a concave region which shows over-segmentation. A concave region may also indicate under-segmentation in which multiple teeth are segmented as a single tooth. In step 704, the code value distribution of one or more reconstructions inside and outside the segmented region are evaluated. In one example embodiment of this invention the reconstruction is a virtual monochromatic reconstruction. A difference in code value distributions may indicate that scan data processing in step 608 was not sufficient to produce a virtual monochromatic reconstruction which is completely free of beam hardening artifacts. In step 706, it is determined if additional scan data processing is necessary. It is possible that convex segmented regions correspond to locations at which a tooth is forming into multiple roots. It is a part of this step to distinguish between concavity due to insufficient artifact removal and variation in tooth shape.

It should be understood that the reconstruction code values can take several forms. The code values may be X-ray attenuation coefficients in units of cm⁻¹. Alternatively, the code values may be in Hounsfield units. Also, as is often the case when truncated projections are reconstructed, the code values may not measure a physical property of the scanned object, but are nevertheless useful for tooth segmentation.

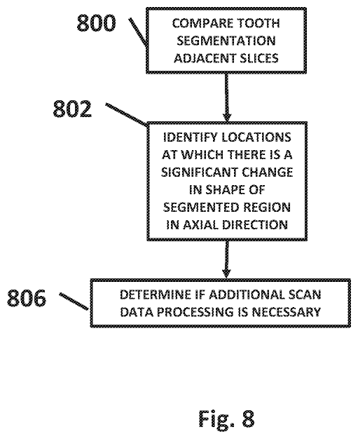

FIG. 8 displays another set of steps that can occur within and form step 613, possibly in parallel with the processing steps in FIG. 7. The steps in FIG. 8 are directed at reducing the under-segmentation problem that is illustrated in image 110 in FIG. 1. In step 800, the tooth segmentation in adjacent axial slices are compared. A large change in segmentation, for example, as measured by the Sorensen-Dice coefficient may indicate that tooth segmentation may be extending into surrounding bone or multiple teeth are segmented as one. In step 802, regions of under-segmentation are identified. In step 806, it is determined if additional processing is necessary. If so, steps 608, 610, and 612 are repeated in such a way to boost material differentiation.

The present invention has been described in detail and with particular reference to example embodiments, but it should be understood that variations and modifications can be affected within the spirit and scope of the invention. The presently disclosed example embodiments are, therefore, considered in all respects to be illustrative and not limiting. The scope of the invention is defined by the appended claims, and all changed or modifications that come within the meaning and range of equivalents thereof are intended to be embraced therein.

What is claimed is:

1. A method for producing a three-dimensional representation of one or more teeth comprising the steps of:
a) using X-ray scans data at two or more different X-ray energy spectra;
b) combining the measured data from the two or more X-ray scans;
c) reconstructing the combined data to form one or more three-dimensional images; and
d) segmenting a tooth in the said one or more three-dimensional images.

2. The method of claim 1, wherein said one or more three-dimensional images has reduced beam hardening artifacts.

3. The method of claim 1, wherein said one or more three-dimensional images has reduced metal artifacts.

4. The method of claim 1, wherein said one or more three-dimensional images has reduced scatter artifacts.

5. The method of claim 1, wherein the scan data is captured with an energy discriminating detector.

6. The method of claim 1, wherein the scan data is captured with an energy discriminating photon counting detector.

7. The method of claim 1, wherein the scan data is captured with X-ray sources with different voltage.

8. The method of claim 1, wherein the scan data is captured with X-ray sources with different filtration.

9. The method of claim 1, wherein the tooth segmentation results are evaluated for the purpose of modifying the combining of data from two or more scans of different X-ray spectra.
